(19) United States
(12) Patent Application Publication
(10) Pub. No.: US 2006/0264508 A1
(43) Pub. Date: Nov. 23, 2006

(54) Modulation of ocular growth and myopia by gaba drugs

(76) Inventor: Richard A. Stone, Havertown, PA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/493,049

(22) PCT Filed: Oct. 2, 2002
(86) PCT No.: PCT/US02/31776

§ 371 Date: Apr. 16, 2004

Related U.S. Application Data
(60) Provisional application No. 60/329,655, filed on Oct. 16, 2001.

(51) Int. Cl. A61K 31/195

(52) U.S. Cl. 514/561

(57) ABSTRACT

Provided are methods and compositions for controlling postnatal ocular growth and the development of ocular errors in the maturing eye of a subject, comprising altering the refraction and/or growth of the maturing eye of a subject by administering to the eye a therapeutically effective amount of at least one GABA drug or compound, including agonists or antagonists (alone or in combination with other compounds), as well as any other drug or composition, regardless of classification, that acts to alter the refractive development and/or growth of the eye. Further provided are methods and compositions for treating or preventing myopia, hyperopia or amblyopia.

### REFERENCE TO RELATED APPLICATION

This application claims priority to U.S. Provisional Application No. 60/329,655, filed Oct. 16, 2001, the content of which is herein incorporated by reference.

### GOVERNMENT INTEREST

This invention was supported in part by Grant No. EY-07354 from the U.S. National Institutes of Health. The Government may have certain rights in this invention.

### FIELD OF THE INVENTION

The present invention relates to the control of postnatal eye growth and myopia. In particular, the invention relates to the effect of γ-aminobutytic acid (GABA) on retinal mechanisms influencing eye development and the influence of drugs and compositions interacting with GABA receptors on eye growth and refractive development.

### BACKGROUND OF THE INVENTION

It has been estimated that about one of every four persons on earth suffers from myopia (commonly known as near-sightedness). At least half of these cases are axial myopia, i.e., an elongation of the eye along the visual axis. At birth, the human eye tends to be relatively short in the axial direction causing a tendency for young children to be hyperopic (commonly known as far-sightedness). During childhood, as the eye grows, the ocular length of the cornea and lens increase and optical properties change. Ideally, no correction is needed for sharp vision at distance and the eye is emmetropic. However, if the eye lengthens too far, distant images focus in front of the plane of the retina and axial myopia results. If, on the other hand, the ocular length of the eye remains too short, near images focus behind the plane of the retina and the result is hyperopia.

Numerous proposed treatments and remedies have been directed at the focussing mechanism at the front of the eye. Largely these have been attempts either to block the focusing ability of the eye (called accommodation) through topical application of drugs or to remove any need for near focus through use of plus lenses that in effect perform the near focus task. Topical drugs that relax the focussing muscle of the eye, the ciliary muscle, are called cycloplegics and have been available for a century.

Recently, significant evidence has been developed showing that myopia results in an eye that is subjected to retinal image degradation. It has been shown that axial myopia can be experimentally induced, in either birds or primates, in an eye in which the retina is deprived of formed images, e.g., by suturing the eye-lids or wearing an image-diffusing goggle (Weisel & Raviola, Nature 266:66(1977)). The experimental myopia induced in primates, such as monkeys, precisely mimics the common axial myopia of humans. Thus, the vision process apparently contributes to the feedback mechanism by which postnatal ocular growth is normally regulated and refractive error is determined in the animal, indicating that the mechanism is neural, and likely originates in the retina.

Convincing evidence has identified a dominant role for the retina in linking postnatal eye growth with visual input (Wallman, Progress in Retinal Research 12:133-153 (1993); Stone, In: Myopia Updates: Proceedings of the 6th International Conference on Myopia, (Tokoro, ed) Tokyo: Springer; 241-254 (1997)). Several retinal neurotransmitters have been implicated in the refractive development and myopia pathogenesis (Stone, 1997; Fischer et al., J. Comp. Neurol 393:1-15 (1998); Fischer et al., Nature Neuroscience 2:706-712 (1999); Fujikado et al., Curr. Eye Res. 16:992-996 (1997); Pickett Seltner et al., Visual Neurosci. 14:801-809 (1997); Stone et al., Proc. Natl. Acad. Sci. USA 86:704-706 (1989); Stone et al., Invest. Ophthalmol. Vis. Sci. 42:557-565 (2001)). Many of the retinal neurotransmitters localize to one or another subtype of retinal amacrine cell. Given that complex, but still poorly defined, characteristics of the visual image-modulated eye growth (Schaeffel et al., Vision Res. 39:1585-1589 (1999)), putative involvement of amacrine cells is consistent with the processing of complex image features in the inner retina (Kolb, Eye 11:904-923 (1997)) and the notion that a multi-component mechanism(s) guides emmetropization. U.S. Pat. Nos. 5,055,302, 5,122,522 and 5,356,892 (Laties and Stone) disclose methods for controlling the abnormal postnatal growth of the eye of a maturing animal using vasoactive intestinal peptide (VIP), PHI or analogues of these peptides or pirenzepine, respectively.

In contrast to the numerous neuropharmacologic drugs that influence experimental myopia, comparatively few have been found to alter the growth and refractive development of eyes with intact visual input, perhaps because vision dependent mechanisms governing eye growth dominate drug effects. For example, dopamine agonists, opiates and basic fibroblast growth factor each inhibit form-deprivation myopia, but none alter growth or refraction of non-occluded eyes (Stone et al., 1989; Rohrer et al., Exp. Eye Res. 58:553-561 (1994); Fischer et al., Visual Neurosci. 15:1089-1096 (1998); U.S. Pat. Nos. 5,284,843; 5,360,801 and 5,571,823). A developmental effect of muscarinic antagonists to reduce growth of non-occluded eyes and induce a refractive shift in the hyperopic direction has been observed in only a single study (Cottriall et al., Exp. Eye Res. 74:103-111 (2002)). Other drugs that have reportedly influenced the growth and refraction of non-goggled eyes of chicks are neurotoxins, such as kainic acid, N-methyl-D-aspartate, tetrodotoxin and others (Stone et al., 2001; Fischer et al., 1998; Wildsoet et al., Invest. Ophthalmol Vis. Sci. 29:311-319 (1998); Ehrlich et al., In: Ciba Foundation Symposium 155: Myopia and the control of eye growth, (Bock; Widdows, eds) Chichester: John Wiley & Sons, pp.63-88 (1990); McBrien et al., Vision Res. 35:1141-1152 (1995); see also U.S. Pat. Nos. 5,637,604 and 5,461,052).

GABA (γ-aminobutyric acid) is a widely distributed inhibitory amino acid neurotransmitter, located in the central nervous system and retina. In the vertebrate retina, GABA localizes to a large and diverse neuronal population (Nguyen-Legros et al., Microsc. Res. Tech. 36:26-42 (1997)), and has been implicated in the signaling of both amacrine and horizontal cells (Kolb, 1997; Barnstable, Curr. Opinion Neurobiol. 3:520-525 (1993); Slaughter, Progress in Retinal and Eye Research 14:293-312 (1995). U.S. Pat. Nos. 5,385,939 and 5,567,731 (Laties and Stone) disclose a composition for the inhibition of the abnormal postnatal axial growth of the eye of a maturing animal which comprises a GABA_{B} receptor antagonist, and a method of alleviating and controlling the development of amblyopia in the eye of a primate animal by administering a γ aminobutyric acid antagonist. However, to date, neither GABA nor its receptors have been reported to be in peripheral nerves to the eye or in the non-retinal tissues of the eye.

The chick, like many other vertebrates, contains in its retina many GABA-based amacrine cells in the inner nuclear layer, horizontal cells and some neurons, in the ganglion cell layer, which likely are displaced amacrine cells, with many nerve fibers in both the inner and outer plexiform layers (Fischer et al, 1998; Agardh et al., Invest. Ophthalmol. Vis. Sci. 27:674-678 (1986); Mosinger et al. Exp. Eye Res. 42:631-644 (1986); Hamassaki-Britto et al., J Comp. Neurol. 313:394408 (1991); Watt et al., Brain Res. 634:317-324 (1994)). Thus, the chick has become an accepted model animal in the art for retina studies, and the findings have proven to be representative for other vertebrates, including humans and other mammals.

GABA receptors traditionally have been classified into three major subtypes: GABA_{A}, GABA_{B} and GABA_{C} receptors (Chebib et al., Clin. Exp. Pharmacol. Physiol. 26:937-940 (1999)). GABA_{A} and GABA_{C} receptors each consist of ligand-gated chloride channels. Most GABA_{A} receptors are believed to be comprised of five subunits from multiple subunit classes (α1-6, β1-4, γ1-3, δ, ε, θ and/or π) (Barnard et al., Phannacol. Rev. 50:291-313 (1998); Barnard, In: Pharmacology of GABA and Glycine Neurotransmission, (Môhler, ed.) Berlin, Springer, pp. 79-99 (2001)). GABA_{C} receptors are comprised of one or more of the three different ρ subunits, which are not known to complex with proteins of the other subunit classes (Barnard et al., 1998; Bormann et al., In: Pharmacology of GABA and Glycine Neurotransmission, (Môhler, ed.) Berlin, Springer, pp. 271-296 (2001)). Despite distinctive pharmacology, structure, genetics and function (Bormannet et al., 2001), GABA_{C} receptors have recently been re-classified as the GABA_{A0r} subtype of the GABA_{A} receptor family (Barnard et al., 1998). Accordingly, the general term “GABA_{A} receptors” are used herein for the large family of bicuculline-sensitive GABA receptors and “GABA_{A0r} receptors” for the bicuculline-insensitive, ρ-containing GABA_{A} receptor subset that had been previously termed “GABA_{C} receptors.”

GABA_{B} receptors are metabotropic, G-protein linked receptors, coupled to adenylaie cyclase or to Ca⁺⁺and K⁺channels. One of the functions of the GABA_{B} receptors is modulation of neurotransmitter and neuropeptide release (Bormann, Trends Pharmacol Sci. 21:16-19 (2000); Bowery In: Pharmacology of GABA and Glycine Neurotransmission, (Môhler, ed.) Berlin, Springer, pp. 311-328 (2001)).

GABA_{A} , GABA_{A0r} and GABA_{B} receptor subtypes are each expressed widely in the vertebrate retina (Lukasiewicz et al., Cell Dev. Biol. 9:293-299 (1998)). In fact, the predominant location of GABA_{A0r} receptors in brain tissues is the neural retina. GABA_{A} receptors occur on both pre-synaptic and post-synaptic locations in many types of retinal neurons. GABA_{A0r} receptors are found mainly, but not exclusively, on bipolar cells. GABA_{B} receptors tend to localize post-synaptically on amacrine and ganglion cells. Available data in chicken conform to these generalities.

By immunohistochemistry, it has been shown that GABA_{A} receptors occur in the outer and inner plexiform layers of the retina and in distinct types of retinal amacrine cell soma (Yazulla et al., J. Comp. Neurol. 280:15-26 (1989)). GABA_{A0r} receptors also localize to both plexiform layers, evidently corresponding to processes of bipolar cells (Koulen et al. J. Comp. Neurol. 380:520-532 (1997). In situ hybridization in chick retina has identified GABA_{A0r} mRNA at retinal levels corresponding to the somata of horizontal, bipolar, amacrine, and perhaps ganglion cells (Albrecht et al., Neurosci Lett. 189:155-158 (1995)). However, to date, there has been no biochemical identification of GABA_{B} receptors, nor have they been localized at a cellular level in the retina.

In the retina, GABA co-localizes and/or interacts with other neurotransmitters that are potentially involved with eye growth control (Stone, 1997; Stone et al., Proc. Natl. Acad. Sci. USA 85:257-260 (1988); Guo et al., Curr. Eye Res. 14:385-389 (1995)), including dopamine (Stone et al., 1989; Nguyen-Legros et al., 1997; Kazula et al., Visual Neurosci. 10:621-629 (1993)), and acetylcholine (Stone et al., 2001; Hamassaki-Britto et al., 1991; Agardh, Acta Physiol. Scand. 126:33-38 (1986); Santos et al., Eur. J Neurosci. 10:2723-2730 (1998); Fischer et al., Brain Res. 794:48-60 (1998); Duarte et al., J Neurosci. Res. 58:475-479 (1999); Neal et al., Visual Neurosci. 18:55-64 (2001)). The first evidence for the involvement of GABA receptors in eye development was U.S. Pat. Nos. 5,385,939 and 5,567,731 ,(Laties and Stone) that disclosed a composition for the inhibition of the abnormal postnatal axial growth of the eye of a maturing animal and which comprises a GABA_{B} receptor antagonist, and a method of alleviating and controlling the development of amblyopia (lazy eye) in the eye of a primate animal by administering a GABA_{B} antagonist. In a subsequent report, many GABA-containing retinal neurons were found to be relatively resistant to quisqualic acid toxicity, and experimental myopia still developed after ocular administration of this neurotoxin. It was then suggested that retinal GABA may be, in some way, relevant to myopic eye growth (Fischer et al., 1998), however, the mere suggestion has, to date, remained unsubstantiated except for the inventor's initial investigation of GABA_{B} receptor antagonists. as stated. As a result, until the present invention there has been no direct added evidence for the role of GABA_{B} receptors in postnatal eye growth control, refractive development or myopia, or elucidation of the involvement of any other GABA receptor subtypes or GABA drug mechanisms, which given the unpredictable nature of biological systems, means that the function of retinal GABA was largely unknown and there remained an unmet need in the art. Moreover, a need also remained for a composition and methods for its use that would affect ocular growth in the postnatal, developing eye in both the axial and equatorial dimensions.

### SUMMARY

The present invention provides direct evidence demonstrating the effect of drugs interacting with γaminobutyric acid (GABA) receptors in the retina that influence eye development, and comprises compositions and methods to control ocular growth and refractive development in the postnatal developing eye, and include control of myopia. In controlled analyses, the eyes of subjects, some of which wore a unilateral goggle to induce myopia and received daily intravitreal injections of agonists or antagonists to the major GABA receptor subtypes were studied by refractometry, as well as ultrasound and caliper measurements to assess the effects of the drugs on eye development.

Antagonists to GABA_{A} or GABA_{A0r} receptors were found to inhibit form-deprivation myopia. GABA_{A} antagonists showed greater inhibition of myopic growth in the equatorial than the axial dimension; a GABA_{A0r} antagonist displayed parallel inhibition in axial and equatorial dimension. When tested, one GABA_{A0r} agonist, but no GABA_{A} agonists, altered the myopic refraction of the goggled eyes in the test animals. GABA_{B} receptor antagonists, more so than a GABA_{B} receptor agonist, also slowed myopia development, inhibiting axial growth more effectively than equatorial expansion of the goggled eyes. Retinal GABA content was shown to be slightly reduced in goggled eyes.

When administered to non-goggled eyes, GABA_{A} and GABA_{A0r}agonists and antagonists also altered eye growth, frequently stimulating it. However, only one GABA_{A} agonist was shown to induce a myopic refraction. Several of these agents stimulated eye growth in the axial, but not in the equatorial dimension. A GABA_{B} agonist and GABA_{B} antagonist also stimulated eye growth, but did not alter refraction.

Therefore, in accordance with the findings of the present invention drugs affecting GABA_{A} , GABA_{A0r} and GABA_{B} receptors modulate eye growth and refractive development in the postnatal eye. The anatomical effects of these drugs on the eye further indicate that eye shape, not simply eye size, is regulated. A retinal site of action conforms with the known ocular localizations of GABA, its receptors, and the altered retinal biochemistry in form-deprived eyes.

It is an object of this invention, therefore, to provide a GABA receptor agonist or antagonist or other compound that effectively alters eye growth and refractive development in young animals or children. This alteration can be inhibition or reversal of myopia, such as by inhibiting the axial elongation or equatorial expansion in myopic eyes by suitable agents. The alteration also can involve stimulation of eye growth and reduction of hyperopia, to inhibit or reverse hyperopia by suitable agents. Also provided is a method for controlling postnatal ocular growth and the development of ocular errors in the maturing eye of a subject, comprising modulating retinal levels of GABA in the maturing eye of the subject by administering to the eye to a therapeutically effective amount of at least one GABA drug or compound, or drug of another class.

Further, it is an object to provide compositions affecting GABA receptors of types GABA_{A}, GABA_{B} or GABA_{A0r} in the retina of the maturing eye; and methods, wherein such compositions are administered preferably as a therapeutically effective amount of at least one agonist of at least one type of GABA receptor in the retina of the eye. In another preferred embodiment, there is provided the administration of a drug or compound comprises a therapeutically effective amount of at least one antagonist of at least one type of GABA receptor in the retina of the eye.

It is also an object to provide methods and compositions of the foregoing, wherein the modulating step comprises inhibiting or reversing myopia in the eye of a postnatal subject. Preferably, axial length or vitreous chamber depth is reduced, along with a corresponding reduction in myopic refraction. In yet another a preferred embodiment, a therapeutically effective amount of GABA_{A} receptor agonist or antagonist is administered to the maturing eye in a carrier or diluent buffered to a pH suitable for ocular administration. Examples of such GABA_{A} receptor antagonists are SR95531 or bicuculline. In an additional preferred embodiment, a therapeutically effective amount of GABA_{A0r} receptor agonist or antagonist is administered to the maturing eye in a carrier or diluent buffered to a pH suitable for ocular administration. One such GABA_{A0r} receptor agonist is CACA, and one such GABA_{A0r} receptor antagonist is TPMPA. In another preferred embodiment, a therapeutically effective amount of GABA_{B} receptor agonist or antagonist is administered to the maturing eye in a carrier or diluent buffered to a pH suitable for ocular administration. One such GABA_{B} receptor agonist is baclofen, and one such GABA_{B} receptor antagonist is CGP46381.

It is also an object to provide methods and compositions of the foregoing, wherein the modulating step comprises inducing ocular growth and reducing hyperopia (the latter, by stimulating a myopic shift in refraction), or a combination thereof, in the eye of a postnatal subject. Preferably, axial length or vitreous chamber depth is enhanced, corresponding to a reduced hyperopic (or increased myopic) refraction, and reducing a tendency towards hyperopia. In a preferred embodiment, a therapeutically effective amount of GABA receptor agonist or antagonist is administered to the maturing eye in a carrier or diluent buffered to a pH suitable for ocular administration. One such GABA_{A} agonist is muscimol; one such GABA_{A0r} antagonist is TPMPA.

It is yet another object of the invention to provide a method for determining the effectiveness of the GABA agents used for controlling for postnatal ocular growth and the development of ocular errors in the maturing eye of an animal.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended figures.

FIGS. 1A-1C graphically depict the drug effects on refractions of goggled eyes—that is, drug activities against myopia. Effects on refraction are shown in FIG. 1A for drugs selective for GABA_{A} receptors, in FIG. 1B for drugs selective for GABA_{A0r} receptors, and in FIG. 1C for drugs selective for GABA_{B} receptors. The comparative controls, goggled chicks receiving vehicle only as treatment, are shown by the bars with cross-hatched markings to distinguish the controls from the other findings. n=number of chicks in each cohort. Data are shown as the difference of goggled minus contralateral control eyes. P-values apply to the use of one-way analysis of variance (ANOVA) on the differences between drug-treated goggled and contralateral vehicle-treated non-goggled eyes., n.s.=not significant.

FIG. 2 graphically depicts the effects of GABA_{A} and GABA_{A0r} selective drugs (angonist and antagonists) on dimensions of the goggled eyes—that is, drug activities in inhibiting the excessive eye growth in myopia. The number of chicks in each experimental group appears in FIG. 1. The comparative controls, goggled chicks receiving vehicle only as treatment, are shown by the bars with cross-hatched markings to distinguish the controls from the other findings. Data are shown as the difference of goggled minus contralateral control eyes. P-values apply to the use of one-way ANOVA on the differences between drug-treated goggled and contralateral vehicle-treated non-goggled eyes. n.s.=not significant.

FIG. 3 graphically depicts the effects of drugs selective to the GABA_{B} receptor on dimensions of goggled eyes eyes—that is, drug activities in inhibiting the excessive eye growth in myopia. The number of chicks in each experimental group appears in FIG. 1. The comparative controls, goggled chicks receiving vehicle only as treatment, are shown by the bars with cross-hatched markings to distinguish the controls from the other findings. Data are shown as the difference of goggled minus contralateral control eyes. P-values apply to the use of one-way ANOVA on the differences between drug-treated goggled and contralateral vehicle-treated non-goggled eyes. n.s.=not significant.

FIG. 4 graphically depicts the drug effects on refraction in non-goggled eyes as indicated. Three drugs are shown that had a refraction effect identified in the overall ANOVA, but only muscimol induced a statistically significant shift in refraction of drug-treated eyes compared to contralateral vehicle-treated eyes. The bars are shaded to distinguish the dosage effects from each other, but in each panel of FIG. 4, the shading is consistent for each dosage level. The P-values shown apply to the use of a two-way repeated measures ANOVA (one factor replication, using eye as the replicated factor) to assess the statistical strength of a drug effect. n.s.=not significant in the drug-treated to contralateral vehicle-only-treated eye comparison. †=effects reached statistical significance in the dose comparison, but not in the drug-treated to vehicle-only-treated eye comparison.

FIG. 5 graphically depicts the drug effects on the dimensions of non-goggled eyes for drugs influencing at least one parameter. The number of chicks in each cohort appear in FIG. 4, as described. The bars are shaded to distinguish the dosage effects from each other, but in each panel of FIG. 5, the shading is consistent for each dosage level. The P-values shown apply to the use of a two-way repeated measures ANOVA (one factor replication, using eye as the replicated factor) to assess the statistical strength of a drug effect. n.s.=not significant. ‡=effects reached statistical significance in the dose-eye interaction only, but not in the drug-treated to contralateral vehicle-only-treated eye comparison.

### DESCRIPTION OF PREFERRED EMBODIMENTS OF THE INVENTION

In the ordinary visual function of the eye of an animal or human, light forming an image passes through the lens and is received by the retina, and the retina transmits the information to the optic nerve, which then sends it on to the brain. Retinal neurochemicals (i.e., neuro-active chemical compounds) are key components in the vision process. Specifically, light forming the image is sensed by the light receptors, the rods and cones, of the retina. In the regular process of transmitting the image information to the brain, retinal nerve cells, in association with the photoreceptors, release neurochemicals and pass electrical signals transmitting information to adjacent retinal cells as parts of a network in the retina leading to the formulation and qualities of the signals to the optic nerve. These photoreceptors act as transducers changing light energy into electrical and/or chemical signals.

When the eye of an animal during its postnatal growth period is deprived of vision (e.g., by fixing a translucent or image distorting goggle over the eye) or otherwise subjected to retinal image degradation, the result ordinarily is abnormal ocular growth leading to myopia. During this period of image deprivation or degradation, it has been found that the metabolism of certain retinal neurochemicals is altered leading to changes in retinal concentrations thereof. Specifically, it has been noted during periods of ocular image deprivation in maturing birds or primates, that chemical alterations take place in the retina concurrent with the excessive ocular growth leading to myopia.

The present invention comprises methods for controlling postnatal ocular growth and the development of refractive errors in the eyes of a young, maturing animal or human by administering to the eye drugs or compositions that interact with GABA receptors. Active drugs act by modulating retinal levels of GABA, which are shown to be reduced in myopia. While the growth responses to GABA drugs are complex, evidence is provided herein demonstrating that GABA receptor agonists or antagonists alter eye growth, influencing both the progression of form-deprivation myopia and the growth of eyes with normal visual input. While the altered retinal concentration of GABA in form-deprived myopic eyes is modest in magnitude, the consistency of the change in the various test animals supports the involvement of retinal GABA-based neurons in eye growth control. Together with the knowledge that GABA is expressed by diverse retinal neurons, and the fact that ocular locations of GABA and its receptors are known, the present-findings further support the principle that the retina modulates eye growth and that retinal GABA can modulate refractive development.

Broadly stated, the development or progression of ocular error disorders, such as myopia, hyperopia, amblyopia or the like in the eye of a postnatally maturing animal can be inhibited by the postnatal ocular control of the presence of a neurochemical, or by an agonist or antagonist of the neurochemical, including circumstances in which the neurochemical is found to be altered under conditions during ocular maturation in a young animal, ordinarily leading to myopia. The prevention or treatment of myopia is accomplished by the administration of the neurochemical, its agonist or its antagonist or other composition that influences eye growth and refractive development. In an alternative, it is also accomplished by the administration of drugs that otherwise interact with the synthesis, storage, release, receptor interaction, reuptake, or degradation of the naturally-occurring neurochemical, thus influencing the tissue levels and/or bioavailability of such naturally-occurring neurochemical, wherein the neurochemical, or its agonist or antagonist, influences the growth and refractive development of myopic or hyperopic eyes.

Despite notable differences in anatomy between the eyes of primates and those of birds, image deprivation-induced myopia (“form-deprivation myopia”) in chickens closely resembles that in the primate, as shown by studies made on chicks and young monkeys. In both species, evidence suggests that control for postnatal ocular growth is substantially local, within the eye, apparently originating at the retina. Because the chicken matures quickly, newborn chicks were used extensively in studies made in connection with this invention.

One useful chick model is the form-deprivation model, in which the vision of one eye is obscured by a goggle or eyelid suture and ipsilateral eye enlargement and myopia results. In this case, form-deprivation myopia is used to identify agents potentially, useful for retarding myopia in children. In the chick model, as described here, some chicks are fitted with a unilateral goggle to induce myopia and received daily intravitreal injections of agonists or antagonists to the major GABA receptor subtypes. The eyes are then were studied by refractometry, and ultrasound and caliper measurements to assess the affects of the drugs on eye development. Retinas of other chicks also wearing a unilateral goggle were assayed for GABA content for comparison purposes.

As the terms are used in this invention, an agonist or antagonist of a neurochemical is a compound that affects the action of the neurochemical in the retinal tissue. An agonist is an agent that activates a receptor, leading to an intracellular response. Thus, agonists mimic the effects of endogenous regulatory compounds. For purposes of this application, an antagonist of the neurochemical is a compound that opposes, or blocks the action of the neurochemical on the retinal tissue, effectively inhibiting the action of an agonist, thereby effectively inhibiting excessive or abnormal postnatal axial growth of the eye of a maturing animal. The antagonist is useful under conditions ordinarily leading to excessive or abnormal axial growth and/or equatorial expansion. Although ocular administration is described herein, and is generally preferred, systemic administration may also be employed under suitable circumstances.

GABA Drug Effects on Form-Deprivation Myopia.

Agents from each class of GABA drugs are shown in various embodiments of the present invention to alter myopia progression. Antagonists, but not agonists, to GABA_{A} receptors show unusual inhibitory activity against form-deprivation myopia. In preferred embodiments of the invention, receptor antagonists, bicuculline and SR95531, each markedly reduce equatorial expansion of the vitreous chamber of the eye beneath a goggle. Neither significantly altered the axial dimensions of goggled eyes, and only SR95531 caused any reduction in the myopic refraction. In any event, GABA_{A} antagonists represent the first class of drugs that have been reported to inhibit the growth of goggled eyes chiefly in the equatorial dimension.

However, in another preferred embodiment of the invention, the GABA_{A0r} receptor antagonist TPMPA was shown to be a much more potent against experimental myopia than the GABA_{A} receptor antagonists. TPMPA largely eliminates the myopic refractive shift and significantly reduces the axial length of the eye and vitreous chamber depth as measured by ultrasound. It also blocked the equatorial expansion of the eye. The GABA_{A0r} receptor agonist CACA exerted a modest, perhaps biphasic effect on the refraction of goggled eyes, but none of the size measurements were altered by CACA.

For GABA_{B} selective drugs, both agonists and antagonists show some degree of anti-myopia activity. The antagonist CGP46381 was the most effective of these drugs, inhibiting myopia and limiting axial vitreous chamber and equatorial expansion.

GABA Drug Effects on Non-Goggled Eyes.

As with goggled eyes, agents from each class of GABA drugs influence the development of non-goggled eyes. In certain embodiments, drugs interacting with GABA_{A} and GABA_{A0r} receptor subtypes proved to be the most effective, and agents selective for GABA_{B} receptors showed much less potent stimulatory effects. In a preferred embodiment of the invention, the mixed GABA_{A} agonist muscimol had the greatest effect on the eye, increasing not only axial and vitreous chamber lengths, but also expanding the equatorial diameter. Muscimol was the only drug tested that induces a statistically significant myopic shift in refraction. Presumably, non-goggled eyes receiving the other drugs remained emmetropic because the optical elements of the eye otherwise compensated for the elongated axial components.

In another embodiment, the GABA_{A} receptor antagonist SR95531 also enhanced axial and vitreous chamber length, but its effects on refraction did not reach statistical significance, and it did not alter the equatorial dimension of non-goggled eyes. Drugs active at GABA_{A0r} receptors also stimulate eye growth, wherein the enhancement is selective for the axial dimension. In one embodiment, the agonist CACA was shown to stimulate axial growth to a modest degree without altering refraction or affecting equatorial diameter.

By comparison, in an alternative embodiment, the GABA_{A0r} receptor antagonist TPMPA stimulates axial elongation and vitreous chamber depth, also without altering refraction. The geometry of the TPMPA effect is unusual, as the equatorial dimension actually diminished in TPMPA-treated non-goggled eyes.

The ability of GABA drugs to stimulate eye growth of non-goggled eyes and induce a refractive shift towards myopia indicates that such agents can also find utility in treating hyperopia or farsightedness. In hyperopia, the eye tends to be relatively short, but stimulating eye growth corrects this problem. The hyperopic (or “plus”) refractive error of farsighted eyes is also reduced or corrected by GABA drugs as the myopic (or “minus”) shift in refraction reduces or neutralizes the hyperopic refractive error. Since the growth and optical effects of hyperopia and myopia are opposite, the “induction of myopia” in open eyes establishes the possibility of treating hyperopia. As hyperopia in children can lead to either strabismus (crossed eyes) and/or amblyopia (lazy eye), this is another application of the invention.

Underlying Pharmacologic Mechanisms.

Because the effects of the GABA (drugs are complex, they do not permit unambiguous interpretation of their underlying pharmacologic mechanisms. In some instances, agonists and antagonists showed analogous growth effects. Even the dose-response curves tended to be complicated, as some of the optimally effective drug doses occurred in the middle of the tested range. U-shaped or inverted U-shaped dose response curves (termed hormesis), have been increasingly recognized in biological responses to drugs Calabrese et al., Trends Pharmacol. Sci. 22:285-291 (2001). Besides issues of bioavailability and other pharmacokinetic considerations, and without intending to be bound to a hypotheses, the inventor has proposed that the ocular responses may reflect the multiplicity of retinal GABA receptor subtypes (Barnard et al., 1998; Barnard, 2001; Bormann et al., 2001).

Moreover, in some instances, biochemical changes to the eye as a result of treatment in accordance with the compositions and/or methods of the present invention may not be detectable by methods currently available. Nevertheless, such changes may still occur and be sufficient to effect control or a change in growth and/or refraction of the eye.

The molecular subunit compositions of retinal GABA receptors have not been extensively characterized; and within the major GABA receptor subgroups, the currently studied drugs could interact with multiple receptor subtypes. Thus, the ocular growth responses to GABA drugs may reflect the complex retinal distribution of GABA receptors, the specific types of GABA receptor subunits in the retina, the interactions of GABA based neurons with other retinal cells involved in eye growth control, and/or differential drug affinities to specific or multiple GABA receptor subunits. Knowledge of the mechanism(s) underlying the invention has no effect on the invention itself.

GABA Drugs and Eye Shape.

Neurotoxin effects on vitreous cavity (Wildsoet et al., 1998; Calabrese et al., 2001), including the vitreous chamber bulge in eyes wearing a hemi-goggle (Wallman, 1993; Wallman et al., Science 237:73-77 (1987)), the action of dopaminergic and muscarinic drugs to inhibit axial elongation, but not equatorial expansion in experimental myopia, (Stone, 1997; Stone, 1989; Stone et al., Exp. Eye Res. 52:755-758 (1991)), and the selective axial elongation of eyes as the initial response in chicks reared under constant lighting (Stone et al., Vision Res. 35:1195-1202 (1995)), each have suggested retinal control of vitreous chamber shape. However, because the growth effects disclosed herein presumably reflect retinal signaling, the ocular responses to GABA drugs similarly implicate retinal control of the ocular form. Depending on the particular drug, and presumably the corresponding receptor subtype, and depending on whether visual input was previously impaired or intact, GABA agents are shown to exert generalized, selective axial or selective equatorial effects on eye shape.

As an example, GABA_{A} and GABA_{A0r} receptors may have distinct roles in modulating eye shape. In goggled eyes, GABA_{A} agonists or antagonists each acted chiefly to inhibit equatorial expansion, but the GABA_{A0r} antagonist TPMPA exerted comparable growth inhibition in both axial and equatorial dimensions. In non-goggled eyes, the GABA_{A} agonist muscimol expanded the vitreous chamber in both axial and equatorial dimensions, but a GABA_{A0r} agonist caused only modest axial lengthening. Also in non-goggled eyes, a GABA_{A} antagonist stimulated axial growth, but a GABA_{A0r} antagonist both stimulated axial growth and inhibited equatorial expansion.

For a clinical perspective on eye shape, selective axial elongation of the vitreous chamber is believed to characterize the ocular morphology of many, but not all, human myopic eyes (Cheng et al., Optom. Vis. Sci. 69:698-701 (1992); Mutti et al., Invest. Ophthalmol Vis. Sci. 41:1022-1030 (2000)). So far, the initial eye growth response to disrupting the dark phase of a 12:12 hour light dark cycle by constant lighting (Stone et al., 1995) and the GABA drug responses comprising the present invention are the only known or published conditions inducing selective axial elongation of the vitreous chamber.

Treatment to inhibit axial-elongation myopia during maturation of an animal can be administered by the use of an effective amount of the agent by intravitreal injection, but for treatment purposes, eye drops, ointments or gels as topical applications or orally administered pills, tablets or liquids are preferred. Indeed, in the vast majority of cases, treatment agents are administered to human eyes by the topical application of medications, typically as eye drops, ointments or gels, but other topical means of drug administration are also accomplished by the present invention. Eye drops are typically prepared at a concentration of active agent ranging from between about 0.1 and 4 percent in an ophthalmic medium. For example, although not intended to be limiting, a 1% solution of the mixed GABA_{A} agonist, muscimol, in a delivery vehicle appropriate for the eye would be a likely concentration for clinical use.

By “effective amount” or “therapeutically effective amount” is meant an amount of GABA drug, alone or with a carrier, diluent, another agonist or antagonist and/or other synergistic component, such that when administered to an animal, preferably a human, it is effective to treat or prevent refractive errors, such as myopia or hyperopia, as demonstrated e.g., by caliper or ultrasound measurements as herein disclosed, or by standard eye exam in a human that could involve ocular refraction, ultrasound or related techniques. Compositions of the invention present the opportunity of obtaining significant reductions in myopia using reduced dosages of; e.g., GABA drugs, thereby diminishing the side effects and possible pain or toxicity, which could result from alternative therapies.

The terms “induced,” “stimulated,” “enhanced,” “increased,” “inhibited,” “prevented” and the like are given their ordinary dictionary meanings with regard to ocular growth and myopia. For example, “enhanced” refers to an increase and/or induction of growth. More specifically, “enhancement” refers to the ability of the drug on the GABA receptor to cause or result in an elongated growth of the eye or eyes of an animal in an axial or equatorial direction as shown. By “reversal” of an ocular error is meant in the case of a myopic eye, decreasing its relative size in at least one parameter, thereby making it less myopic (or more hyperopic); or in the case of a hyperopic eye, increasing size or stimulating growth in at least one parameter, thereby making it less hyperopic (or more myopic).

Some constraints in formulation may exist, having to do with pH, preservation and/or stability. A pH of about 6.5 is expected to be acceptable as an ophthalmic drop. Buffering is common for eye drops, and may be necessary with GABA_{A} , GABA_{B}, or GABA_{A0r} or receptor agonists or antagonists. Other additives and ingredients may be present, e.g. those disclosed in Chiou, U.S. Pat. No. 4,865,599 (incorporated herein by reference).

Common regimens for administering eye drops vary from one time a day to 4 times a day spaced evenly throughout waking hours. More effective agents may require fewer applications, or enable the use of more dilute solutions in the eye. Alternatively ointments, gels, solid inserts and local depositors of powders or other formulations are now becoming recognized in clinical practice. Their use avoids problems of drug decomposition and can improve compliance, while at the same time delivering a defined amount of drug. It is, of course also possible to administer the above-described active agents in therapeutically effective amounts and dosages by pills, capsules, liquids, elixirs or other preparations for systemic administration.

By “subject” is meant any bird or animal on which the present invention may be used, or on which it is effective to modulate or prevent ocular error. By “animal” is meant any recognized animal, including wild or commercially valuable species and veterinary animals, as well as primates and humans. It further includes newborn, children, youths or adults, although developing or maturing eyes are preferably those of newborns or young children of any species.

In experiments utilizing animals, such as those mentioned herein, in which axial myopia has been experimentally induced by depriving the retina of formed images, it has been noted by others in primates that amblyopia was also experimentally and coincidentally induced. Amblyopia is evidenced by poor visual acuity in the eye, resulting in poor visual performance. Normally, visual acuity improves during maturation. It is known that amblyopia may occur in humans from unknown causes, or as part of strabismus (e.g. lazy eye), especially in far-sighted children with small eyes. It is likely that administration of a therapeutically effective amount of a GABA drug will also prevent, inhibit or reverse the development of permanent or persistent amblyopia in maturing humans. It is also likely that humans who have already developed amblyopia from other, or even unknown, causes might be aided by similar therapeutic treatment with the aforementioned agents.

The affinity and relative affinity of GABA agonists or antagonists for GABA_{A}, GABA_{B}, or GABA_{A0r} receptors can be determined by means known in the art.

In chicks, as well as in humans, axial elongation and/or equatorial expansion can be documented by comparing the matched eyes of one animal with the eyes of another animal, or by unilaterally treating one eye of the animal with the test drug(s) or compound(s), while treating the other eye with only the drug vehicle as a control, or leaving it untreated. In particular, detecting the GABA effect of drugs used to induce or inhibit axial growth of the eyes of an animal comprises contacting the one eye or one animal's eyes with an agonist or antagonist of the GABA_{A}, GABA_{B}, or GABA_{A0r} receptors, and detecting the change in the axial and or equatorial growth of the eyes, then contacting the other eye or eyes of the control animal with the control agent or vehicle alone used to transport the drug, and measuring the axial and/or equatorial growth of the eyes. Then the axial and/or equatorial growth of the treated eye or the eyes of the animal treated with the drug are compared with the control or vehicle-only eye or those of the animal treated with the control agent. Refractory effects are similarly evaluated. The comparisons are further evaluated by including in the acquired data the effects of goggled eyes versus non-goggled, open eyes.

It is possible that the same neurochemical process described herein, perhaps in different direction and/or degree, is involved in the diminished postnatal ocular axial growth resulting in hyperopia. It is suggested, therefore, that similar excesses or deficiencies of retinal neurochemicals are involved during hyperopia development. As a consequence, treatment for hyperopia can involve the administration of effective amounts of the GABA drug(s).

The case of the present invention lies in the discovery that topical local application of a compound to a normally seeing eye of a young chick can enhance eye growth. The degree of growth enhancement, in turn, is susceptible to modulation by yet other pharmacological agents. The growth effect of the GABA agents can be inhibited by co-administration of agonists or antagonists of the GABA_{A} , GABA_{B}, or GABA_{A0r} receptors, as shown by the effects on the open eye models of the present invention.

The present invention is further described in the following examples. These examples are not to be construed as limiting the scope of the appended claims.

### EXAMPLES

The following experiments were conducted to provide direct evidence for a role of retinal GABA mechanisms in the control of postnatal eye growth.

White leghorn chicks (Truslow Farms, Chestertown, Md.) were reared under a 12 hour light:dark cycle as described by Stone et al., 2001. Chicks were anesthetized with inhalation ether for all goggle applications and drug injections. The experimental eye was the right eye in half of the chicks, and the left eye in the other half of the chicks, assigned in an alternating order within each group. Form-deprivation myopia was induced by attaching a unilateral translucent white plastic goggle to the periorbital feathers with cyanoacrylate glue. All research conformed with the ARVO Resolution on the Use of Animals in Ophthalmic and Vision Research.

Intraocular Drug Administrations.

All goggle applications and/or drug injections began when the chicks reached one week of age. At about four hours into the light phase, a 10 μl intravitreal injection of either drug+vehicle or vehicle alone was given under aseptic conditions to the goggled or experimental non-goggled eye, with all contralateral eyes concurrently receiving vehicle injections.

After four days of treatment the chicks were anesthetized with an intramuscular mixture of ketamine (20 mg/kg) and xylazine (5 mg/kg) for ocular examinations. On this day, the animals received no intraocular injections. Ocular refractions and A-scan ultrasonography were performed as described by Stone et al., Vision Res. 35:1195-1202 (1995). While still under general anesthesia, the chicks were decapitated; and the axial and equatorial dimensions of enucleated eyes were measured with digital calipers. As the coronal profile of the chick eye is elliptical, the equatorial dimension was reported as the mean of the shortest and longest equatorial dimensions.

Table 1 lists the studied drugs, their characteristic affinity(ies) to GABA receptor subtype(s), the suppliers and the ranges of daily doses in μg. The daily doses administered in specific experiments are provided in the Figures and in the described Results, below.

TABLE 1
Drugs, Activity and Dose Ranges.

| | | | ranges of injected drug doses | | |
|---|---|---|---|---|---|
| | Pharmacologic | Chemical name; and drug | (μg); calculated peak vitreous | | |
| | Activity# | supplier^{†} | levels (μM)* | | |
| GABA_{A} | | | | | |
| drugs | | | | | |
| Muscimol | Mixed | Muscimol hydrobromide^{R} | 5-200 μg; | 320-6,410 | μM |
| | GABA_{A} and | | | | |
| | GABA_{A0r}, | | | | |
| | agonist | | | | |
| TACA | Mixed | Trans-4-aminocrotonic acid¹ | 10-100 μg; | 618-6,180 | μM |
| | GABA_{A} and | | | | |
| | GABA_{A0r} | | | | |
| | agonist | | | | |
| Bicuculline | Antagonist | (-)-bicuculline methobromide^{R} | 0.01-50 μg; | 0.135-676 | μM |
| SR95531 | Antagonist | 6-imino-3-(4-methoxy- | 1-100 μg; | 17.0-1,700 | μM |
| | | phenyl)-1(6H)- | | | |
| | | pyridazinebutanoic acid | | | |
| | | hydrobromide^{T} | | | |
| GABA_{A0r} | | | | | |
| drugs | | | | | |
| CACA | Agonist | cis-4-aminocrotomc acid^{R} | 10-200 μg; | 618-12,360 | μM |
| TPMPA | Antagonist | (1,2,5,6-tetraliydro- pyridine- | 0.1-200 μg; | 3.89-7,760 | μM |
| | | 4-yl) methyiphosphinic acid^{R} | | | |
| GABA_{B} | | | | | |
| drugs | | | | | |
| Baclofen | Agonist | R(+)-baclofen^{R} | 10-100 μg; | 250-2,500 | μM |
| CGP46381 | Antagonist | (3-aminopropyl)(cyclo- | 1-200 μg; | 28.5-5,701 | μM |
| | | hexylmethyl)phosphinic acid^{T} | | | |
| SCH50911 | Antagonist | (+)-(2S)-5,5-dimethyl-2- | 10-200 μg; | 361-7,217 | μM |
| | | morpholineacetic acid^{T} | | | |
| 2OH-saclofen | Antagonist | 2-hydroxysaclofen^{R} | 10-200 μg; | 235-4,700 | μM |
| CGP35348 | Antagonist | (3-aminopropyl)(diethoxy- | 1-500 μg; | 27.8-13,880 | μM |
| | | methyl)phosphinic acid^{T} | | | |

#Chebib et al., 1999; Bormann et al., 2001; Bormann, 2000; Bowery, Annu. Rev. Pharmacol. Toxicol. 33: 109-147 (1993); Bolser et al., J. Pharinacol. Exp. Ther. 274: 1393-1398 (1995); Froesti et al., In: Perspectives in Receptor Research, (Giardina et al., eds) Amsterdam: Elsevier Science B.V, pp. 253-270 (1996); Johnston, Trends Pharmacol. Sci. 17: 319-323 (1996); Uchida et al., Eur. J. Pharmacol. 307: 89-96 (1996).
*Calculated maximal post-injection vitreous concentration, assuming initial acute drug with vehicle distribution into 150 μ1 of liquid vitreous (Rohrer et al., Visual Neurosci. 10: 447-453 (1993)).
^{t}Chemical supplier: ^{R}RBI/Sigma (Natick, MA); ^{T}Tocris Cookson (Ballwin, MO).

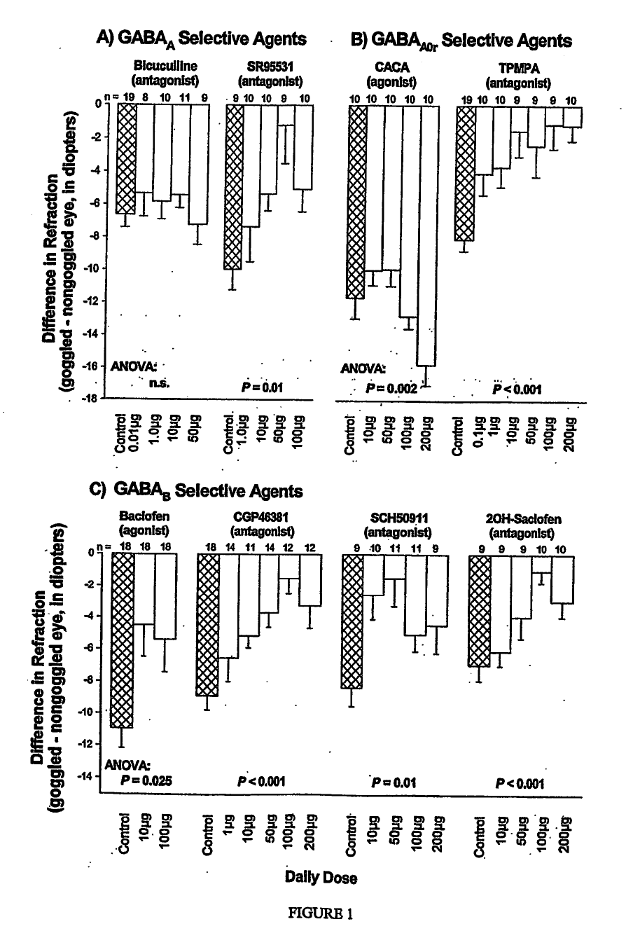

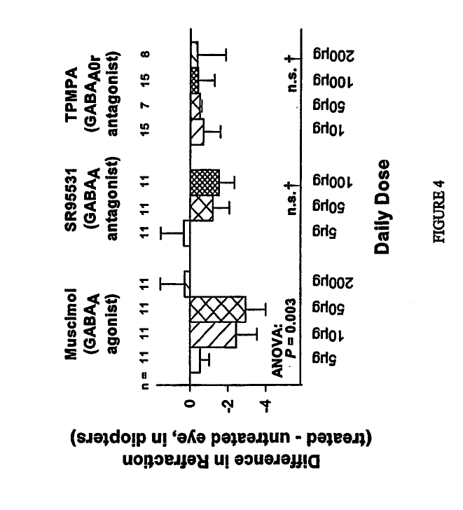

Table 1 also provides an estimate of the maximum drug concentration in μM potentially achievable in the vitreous humor, based upon the assumptions of rapid and uniform drug distribution into a liquid vitreous volume of 150 μl (Rohrer et al., 1993). Comparable eye drop dosages can be calculated accordingly. The number of chicks studied at each drug dose is shown in FIGS. 1 and 4 and in the described Results, below.

Biochemical Assays.

To assay retinal GABA (Allison et al., Anal. Chem. 56:1089-1096 (1984) the same goggle type was placed over one eye of day-old chicks. At 2 weeks of age, the chicks were decapitated after 4 hours into the light cycle; the eyes were enucleated immediately, chilled in iced saline, measured in axial and equatorial dimensions with calipers to confirm ocular expansion and were dissected on ice as quickly as possible. The retinas were immediately frozen and stored individually in liquid nitrogen.

At the time of assay, each frozen retina was placed in 0.5 ml of 0.1 M HClO₄ with 0.3 mM 5-aminovaleric acid HCl as an internal standard at 4° C. and homogenized. The homogenate was centrifuged at 4° C. for 15 minutes at 14,000 rpm, and the supernatant was filtered using an Acrodisc 13mm syringe filter with a 0.2 μm nylon membrane (Gelman Sciences, Ann Arbor, Mich.).

For derivatization, 0.02 ml of the filtered supernatant was incubated for 6 minutes at room temperature with 0.18 ml of 15% carbonate buffer (pH 9.6) containing 5 mM o-phthalaldehyde (OPA; Sigma-Aldrich, St. Louis, Mo.), 50% methanol and 5 mM 2-methyl-propanethiol (Sigma-Aldrich). 25 μl of the derivatized sample was separated on a Beckman Ultrasphere C₁₈ reversed phase (ODS, 5 μm, 4.6 mm×25 cm) column using a high-pressure liquid chromatography system with a LC-4C electrochemical detector (BioAnalytical Systems, West Lafayette, Ind.). The column was eluted a with a mobile phase of 58% 0.1 M Na acetate (pH 5.0) and 42% acetonitrile, with a flow rate of 1.0 ml/minute, and read by the detector with a glassy carbon working electrode at +0.7 V versus an Ag/AgCl reference electrode.

To assay protein, the centrifugation pellet was dissolved in 1.0 ml of 1.0 M NaOH; 10 μl was measured using the Bio-Rad Protein Assay Kit (Bio-Rad Laboratories, Hercules, Calif.) with bovine serum albumin as a standard, following the manufacturer's instructions. GABA levels are reported as μg/mg protein.

Data Analysis.

For goggled chicks, the primary measure of outcome was the drug effect on the ocular response to wearing a goggle, and comparisons of individual doses to each other and to vehicle-treated controls for each drug. Differences in refraction and each size measurement between goggled and contralateral eyes were assessed by one-way analysis of variance (ANOVA). For non-goggled chicks, the primary outcome measure was comparison of drug-treated to contralateral vehicle-treated eyes using a two-way repeated measures ANOVA (one factor replication, using eye as the replicated factor) for refractions and measurements.

The statistical outcomes with the two-way ANOVA are mainly described by the drug-treated to contralateral eye comparison. In the instances for which this comparison did not reach statistical significance, but the overall dose or eye-dose interaction terms yielded a P<0.05, these other comparisons were identified as an alternative indication of potential drug activity. When the ANOVA assumptions of normality or equal variance were not met, the corresponding ANOVA on ranks was used. If the ANOVA identified a treatment effect, the Tukey test for post hoc multiple pairwise comparisons was used to identify specific treatment groups, assuming P<0.05 for statistical significance (Glantz, Primer of Biostatistics, 4^{th} Edition, New York, McGraw-Hill, pp 97-98 (1997)). The Figures show the P values when the overall ANOVA reached a significance level of at least P<0.05, and the post-hoc tests for within group comparisons appear in the Tables that follow.

Retinal levels of GABA in goggled and contralateral non-goggled eyes were compared by student=s paired t-test. Data on anterior chamber depth and lens thickness are not reported for most experiments as these parameters were unaffected in almost all cohorts. The only exceptions, described below, were the lens and anterior chamber for non-goggled chicks receiving CACA, and the lens for non-goggled chicks receiving CGP46381. Results from different cohorts of chicks tested with the same drug, along with the corresponding vehicle-treated controls, were pooled for analysis. Data are shown as mean±S.E.M. and were analyzed with SigmaStat (SPSS, Inc. Chicago, Ill.).

TABLE 2
Post hoc Pair-wise Comparisons of Drug Effects on Goggled Eyes - Tukey Test.

| | | | | | vitreous chamber | Equatorial |
|---|---|---|---|---|---|---|
| | | | axial length | | depth | diameter |
| Drug | Specificity | refraction | ultrasound | calipers | (ultrasound) | (calipers) |
| Bicuculline | GABA_{A} | n.s. | n.s. | n.s. | n.s. | 10 μg vs control |
| | antagonist | | | | | 10 μg vs 0.01 μg |
| SR95531 | GABA_{A} | 50 μg vs. | n.s. | n.s | n.s. | 100 μg vs control |
| | antagomst | control | | | | & 1 μg |
| | | | | | | 50 μg vs control |
| | | | | | | & 1 μg |
| | | | | | | 10 μg vs control |
| | | | | | | & 1 μg |
| CACA | GABA_{A0r} | 200 μg vs. | n.s. | n.s. | n.s. | n.s. |
| | agonist | 50 μg | | | | |
| | | & 10 μg | | | | |
| TPMPA | GABA_{A0r} | 200 μg vs | 100 μg vs | n.s. | 200 μg vs control | 200 μg vs control |
| | antagonist | control | control | | 100 μg vs control | 1 & 0.1 μg |
| | | 100 μg vs | 10 μg vs | | 10 μg vs control | 100 μg vs control, |
| | | control | control | | 1 μg vs control | 10, 1 & 0.1 μg |
| | | 50 μg vs | | | | 50 μg vs control |
| | | control | | | | |
| | | 10 μg vs | | | | |
| | | control | | | | |
| Baclofen | GABA_{B} | 10 μg vs | n.s. | n.s. | n.s. n.s. | |
| | agonist | control | | | | |
| CGP46381 | GABA_{B} | 200 μg vs | 100 μg vs | 100 μg vs | 200 μg vs control | 200 μg vs control |
| | antagonist | control | control | control | 100 μg vs control | 100 μg vs control |
| | | 100 μg vs | | 10 μg vs | & 1 μg | |
| | | control & | | control | | |
| | | 1 μg | | | | |
| | | 50 μg vs | | | | |
| | | control | | | | |
| SCH50911 | GABA_{B} | 50 μg vs | n.s. | n.s. | n.s. | n.s. |
| | antagonist | control | | | | |
| 2OH- | GABA_{B} | 100 μg vs | n.s. | n.s. | n.s. | n.s. |
| saclofen | antagonist | control & | | | | |
| | | 10 μg | | | | |

n.s., P ≧ 0.05 by ANOVA.

The statistically significant post hoc pair-vise comparisons between treatment groups (defined as P<0.05 by the Tukey test) are shown for each drug for which a one-way ANOVA identified a treatment effect (see FIGS. 1-3 for overall ANOVA results).

TABLE 3
Post Hoc Pair-wise Comparisons of Drug Effects on Non-goggled Eyes - Tukey Test

| | | |
|---|---|---|
| | | Equatorial |
| axial length | vitreous chamber | diameter |

| Drug | specificity | refraction | ultrasound | calipers | depth (ultrasound) | (calipers) |
|---|---|---|---|---|---|---|
| Muscimol | GABA_{A}/ | 50 & 10 μg | 200, 50, | 200, 50 & | 200, 50 & 10 μg | 200, 50 & 10 μg |
| | GABA_{A0r} | | 10 & 5 μg | 10 μg | | |
| | agonist | | | | | |
| SR95531 | GABA_{A} | † | ‡ | 100 μg | 100 & 50 μg | n.s. |
| | antagonist | | | | | |
| CACA | GABA_{A0r} | n.s. | 50 μg | n.s. | n.s. | n.s. |
| | agonist | | | | | |
| TPMPA | GABA_{A0r} | § | 10 μg | 200 & | * | 200 & 100 μg |
| | antagonist | | | 100 μg | | |
| Baclofen | GABA_{B} | n.s. | n.s. | * | 100 μg | n.s. |
| | agonist. | | | | | |
| CGP46381 | GABA_{B} | | n.s. | n.s. | 100 μg | n.s. |
| | antagonist | | | | | |

P < 0.05 by ANOVA for drug-treated vs. contralateral eyes, but no specific pair-wise comparison identified by the Tukey test.
P < 0.05 by ANOVA for overall dose effect, but no significant effect identified for drug-treated vs. contralateral eyes; the Tukey test identified the 5 and 100 μg doses as different from each other both overall and also within the treated eyes.
P < 0.05 by ANOVA only for the interaction of eye and dose effects; Tukey test identified drug-treated vs. contralateral eye as significantly different for the 100 μg dose.
P < 0.05 by ANOVA for overall dose effect, but no significant effect identified for drug-treated vs. contralateral eyes; no specific pairwise comparison identified by the Tukey test.
n.s., P ≧ 0.05 by ANOVA

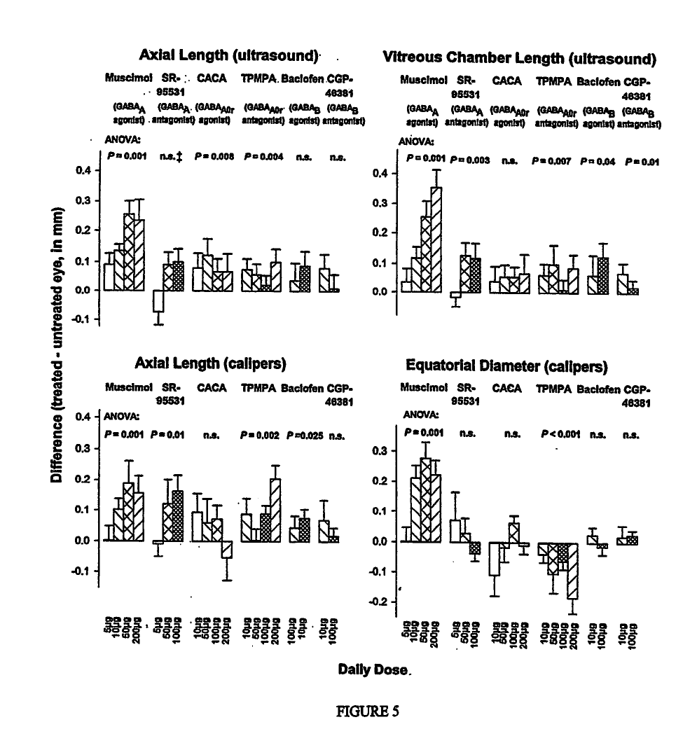

The statistically significant post hoc pair-wise comparisons between drug-treated and contralateral vehicle-treated eyes (defined as P<0.05 by the Tukey test) are identified by each drug dose for which a two-way repeated measures ANOVA identified a treatment effect (see FIGS. 4 and 5 for overall ANOVA results).

Results

Goggled Eyes, GABA_{A} Agents.

GABA_{A} agonists had no effect on form deprivation myopia. The mixed GABA_{A}/partial GABA_{A0r} agonist muscimol did not affect refraction, ultrasound or caliper measurements of goggled eyes (daily doses of 10, 50, 100 or 200 μg; n =9-10/group; data not shown). The mixed GABA_{A}/GABA_{A0r} agonist TACA also had no statistically significant effects on refraction or size measurements when administered to goggled eyes (daily doses of 10 or 100 μg; n=10-13/group; data not shown).

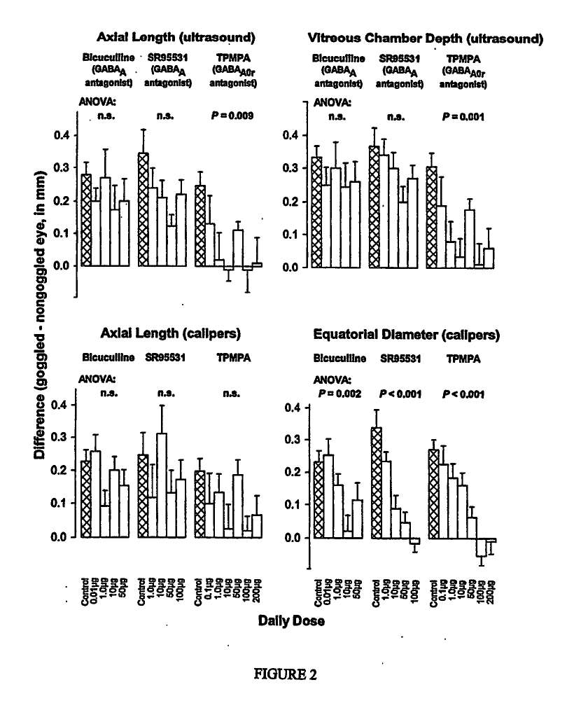

The GABA_{A} antagonists primarily limited equatorial expansion of goggled eyes, as assessed by calipers. The classic GABA_{A} antagonist bicuculline in daily doses up to 50 μg had no effect on the myopic refraction or axial measures of goggled eyes, by either ultrasound or calipers (FIGS. 1A and 2). However, it did reduce the equatorial diameter of goggled eyes (FIG. 2; Table 2). Higher daily doses of bicuculline could not be tested because 100 or 200 μg doses caused retinal whitening, interpreted as gross retinal edema or other toxicity.

Similarly, the GABA_{A} antagonist SR95531 caused pronounced and dose dependent inhibition of equatorial expansion in goggled eyes (FIG. 2; Table 2). SR95531 also reduced the myopic refraction of goggled eyes, wherein the 50 μg daily dose was the most effective (FIGS. 1A; Table 2). While the direction of the trends of SR95531 to reduce axial length or vitreous chamber depth corresponded to the reduced myopic refraction (FIG. 2), none of the length measurements reached statistical significance (P=0.1 or greater). Yet, unlike bicuculline, at the doses used, SR95531 did not cause any ocular toxicity detectable during in vivo ocular examinations or by inspection of the bisected enucleated eyes.

Goggled Eyes, GABA_{A0r} Agents.

The selective GABA_{A0r} agonist CACA had a biphasic effect on the refractive response of an eye to a goggle. The CACA effect on refraction was relatively modest compared to the magnitude of refractive changes in form-deprivation myopia (FIG. 1B), and was not accompanied by any statistically identifiable changes in axial measurements by ultrasound or calipers (data not shown). Perhaps this was because any small corresponding change in axial dimensions was obscured by measurement variability. Moreover, CACA caused no change in the equatorial dimension of the eyes beneath goggles (data not shown).

By comparison, the GABA_{A0r} antagonist TPMPA demonstrated potent anti-myopia effects (FIGS. 1, 2; Table 2). In goggled eyes, it reduced the myopic refraction, blocked the axial and vitreous chamber elongation as measured by ultrasound, and the equatorial expansion as measured by calipers. Any effects on axial growth as measured by calipers, did not reach statistical significance.

Goggled Eyes, GABA_{B} Agents.

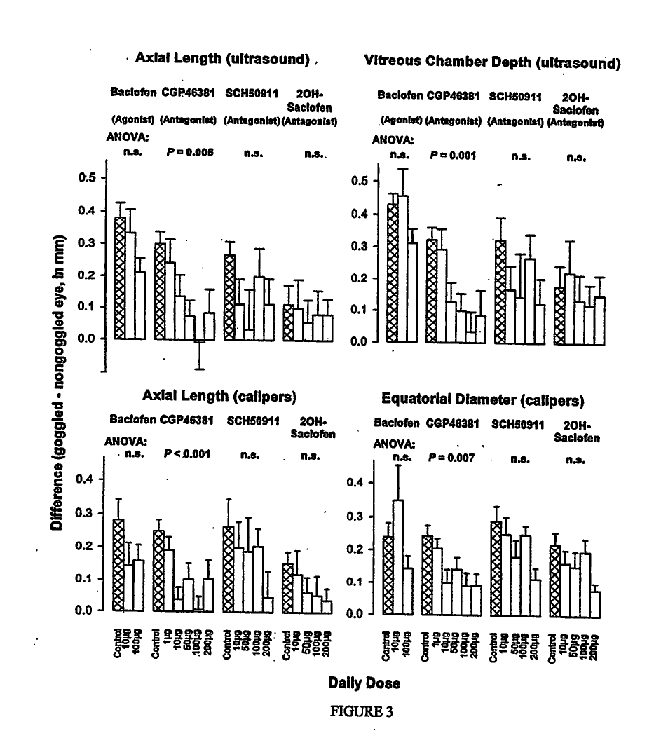

The GABA_{B} agonist baclofen had only a weak anti-myopia effect. It partially reduced the myopic refractive error in the eyes beneath goggles (FIG. 1C, Table 2), but neither the ultrasound nor the caliper measurements revealed statistically significant growth inhibition at the two doses tested (FIG. 3).

By comparison, the high affinity GABA_{B} antagonist CGP46381 demonstrated potent anti-myopia effects (FIGS. 1C and 3; Table 2). It inhibited the myopic refractive shift, the axial and vitreous chamber elongation, and the equatorial expansion of the eyes beneath goggles. In goggled eyes, two other GABA_{B} antagonists SCH50911 and 2OH-saclofen, each reduced the myopic refractions, but none of the tendencies of either drug to reduce ocular dimensions by ultrasound or calipers reached statistical significance (FIGS. 1C and 3). The GABA_{B} antagonist CGP35348 had no statistical effect on refraction or excessive growth of goggled eyes measured by ultrasound or calipers (n=9-18 group; data not shown).

Non-Goggled Eyes, GABA_{A} Agents.

In contrast to its lack of effect on goggled eyes, the GABA_{A} agonist muscimol shifted the refraction to myopia in non-goggled eyes (FIG. 4; Table 3), with a maximum effect at the 50 μg dose. Consistent with this refractive effect, muscimol stimulated axial growth as measured by either ultrasound or calipers, and deepened the vitreous chamber. It also increased the equatorial diameter of non-goggled eyes (FIG. 5; Table 3). An effect on the contralateral vehicle treated eyes was detected with the GABA_{A} agonist muscimol in non-goggled chicks (ANOVA: P<0.05), which substantiates its ability to induce a myopic refractive shift. The refractions of contralateral vehicle-treated eyes varied with the muscimol dose: 5 μg (0.86±0.58, D), 10 μg (+0.11±0.82 D), 50 μg (1.78±1.21 D) and 200 μg (3.82±0.83 D), wherein the 10 and 200 μg doses were statistically different from each other by the Tukey test (“D”=diopters, a unit of refraction, wherein “minus” values signify myopia and “plus” values signify hyperopia). Presumably, since the data are illustrated by normalizing to the contralateral eye (FIG. 4), this degree of myopia in the contralateral eyes accounts for the apparent loss of the myopic refractive shift at the 200 μg dose. There was no evidence for a growth effect in contralateral eyes in any ultrasound or caliper parameter in non-goggled muscimol-treated chicks; and the growth effect of muscimol on treated eyes was not lost at the 200 μg dose (FIG. 5), reinforcing the conclusion that muscimol stimulates growth of non-goggled eyes and induces myopia.

The mixed GABA_{A} agonist TACA, however, was found to have influenced neither refraction nor ocular size measurements of the drug-treated eyes when compared to their contralateral vehicle-only-treated control eyes (daily doses of 10 or 100 μg; n=10/group; data not shown).

The GABA_{A} antagonist SR95531 also stimulated eye growth (FIG. 5, Table 3), but somewhat less effectively than muscimol. Compared to vehicle-treated contralateral eyes, SR95531 enhanced axial growth as measured by calipers and lengthened the vitreous chamber by a comparable amount. While not significant for the drug-treated versus the contralateral eye comparison, the ultrasound measurements of axial length did reveal significant eye lengthening in the dose comparison (P=0.02). The Tukey test identified treated and control eyes as significantly different for the 100 μg drug dose. For refraction, an overall dose effect (P=0.046) was seen, but the myopic shift of up to 1-2 diopters in the drug-treated eyes when compared to contralateral eyes, did not reach statistical significance (P=0.07). SR95531 had no significant effect on equatorial diameter of non-goggled eyes. Given the potential for retinal toxicity (see above), bicuculline was not tested against the non-goggled eyes.

Non-Goggled Eyes (Including Anterior Chamber and Lens Effects), GABA_{A0r} Agents.

While not affecting refraction (FIG. 4 or data not shown), the selective GABA_{A0r} agonist CACA slightly stimulated axial length by ultrasound (FIG. 5; Table 3; n=9-10/group), with a trend for increased vitreous chamber length (FIG. 5; P=0.056). CACA did not affect lens thickness or anterior chamber depth for the primary outcome comparison (see above methods) of drug-treated to vehicle-treated eyes (P=0.22 for lens; P=0.80 for anterior chamber), but it did exert a statistically significant effect on both the lens and anterior chamber in the overall dose comparison (P=0.001 for lens; P≦0.001 for anterior chamber). In this regard, chicks receiving the 10 μg dose differed from cohorts receiving other doses by having thinner lenses and deeper anterior chambers overall. For the lens, the Tukey test identified the overall lens thicknesses of chicks receiving the 10 μg dose as statistically different from chicks receiving either the 100 or 200 μg doses. In chicks receiving the 10 μg dose, the lenses of the drug- and vehicle-treated eyes each measured 2.22±0.02 mm. The lenses of drug-treated eyes of chicks receiving the 10 μg dose were 0.16 mm thinner than lenses at the 100 or 200 μg doses, and the contralateral vehicle-treated eyes of chicks receiving the 10 μg dose were 0.10 mm thinner than lenses of vehicle-treated eyes from the other two cohorts.

The Tukey test identified the differences in drug-treated, but not in vehicle-treated eyes as statistically different for within-eye comparisons. For the anterior chambers, the Tukey-test identified anterior chambers of chicks receiving the 10 μg dose as statistically different from those of chicks receiving the 50, 100 or 200 μg doses. At the 10±μg dose, the anterior chambers of the drug- and vehicle-treated eyes measured 1.32±0.03 and 1.31±0.03 mm, respectively, and the anterior chambers of drug- and vehicle-treated eyes of chicks receiving the 10 μg dose were some 0.08-0.14 mm deeper than those of eyes at the higher doses. For within-eye comparisons, the Tukey test identified the 10 μg dose as statistically different from the 100 and 200 μg doses in drug-treated eyes, and the contralateral vehicle-treated eyes of chicks receiving the 10 μg dose as statistically different from contralateral eyes of those receiving the 200 μg dose. No other growth measures reached statistical significance (FIG. 5).

When administered to non-goggled eyes, the GABA_{A0r} antagonist TPMPA stimulated both axial growth and vitreous chamber length (FIG. 5; Table 3) by a modest degree. In contrast to the axial dimensions, TPMPA reduced the equatorial diameter of non-goggled eyes. As with a number of drugs given to non-goggled eyes, the slight myopic refractive shift reached statistical significance in the dose (P=0.02), but was not found in the drug-treated versus the contralateral eye (P=0.14) comparisons.

Non-Goggled Eyes, GABA_{B} Agents.

When administered to non-goggled eyes, the GABA_{B} agonist baclofen caused modest deepening of the vitreous chamber. A comparable amount of axial elongation reached statistical significance when measured by calipers, but not when measured by ultrasound (FIG. 5; Table 3; n=8 at each dose). None of the other effects of baclofen on the non-goggled eyes, including refractions (data not shown), reached statistical significance.

The most effective GABA_{B} antagonist against myopia, CGP46381, was also tested at two daily doses to determine the effect on non-goggled eyes (n=10/group). CGP46381 slightly enhanced vitreous chamber length in non-goggled eyes (FIG. 5; Table 3). Comparable increases in axial length by ultrasound or caliper measurements did not reach statistical significance.

While CGP46381 had no influence on lens thickness comparing the drug- to vehicle-treated eyes (ANOVA:P=0.58), it did exert a statistically significant lens effect in the dose comparison (P=0.03), indicating that overall the lens thicknesses of chicks receiving the 10 μg dose were statistically different from chicks receiving the 100 μg dose. In chicks receiving the 10 μg dose, the lenses of the drug- and vehicle-treated eyes each measured 2.27±0.04 and 2.26±0.03 mm, respectively. These measurements were 0.07 and 0.06 mm thicker, respectively, than the lenses of the drug- and vehicle treated eyes respectively of chicks receiving the 100 μg dose.

For the within-eye comparisons, the Tukey test identified these differences within the drug-treated, but not within the vehicle-treated eyes, as statistically significant. CGP46381 did not exert statistically identifiable changes in refraction (data not shown), anterior chamber depth (data not shown) or equatorial diameter (FIG. 5).

Retinal Biochemistry.

By HPLC-ED assay, GABA levels in the non-goggled eyes were measured to have 10.8±0.2 μg/mg protein, which remained consistent with published values in the chick retina (Nistico et al., Res. Commun. Chem. Pathol. Pharmacol. 40:29-39 (1983)). GABA levels in contralateral goggled eyes were measured to have 10.3±0.2 μg/mg protein. While the magnitude of this difference is small, the reduction in retinal GABA of myopic eyes did reach statistical significance (N=23 pairs of eyes; P<0.02, two-tailed student's paired t-test).

In Summary.

GABA drugs both inhibit form-deprivation myopia and influence the growth of eyes with normal visual input, thus identifying GABA receptors in the mechanism that modulates eye growth and refractive development. Both ion channel-gated receptors (GABA_{A} and GABA_{A0r} or receptors) and G-protein-linked receptors (GABA_{B} receptors) are implicated by the drug responses. The complex anatomical effects of these drugs reinforce the fact that retinal mechanisms modulate the shape, and not just the overall size, of the developing eye. A site of action at the neural retina is consistent with the known ocular localizations of GABA and its receptors, with the small but consistent reduction in retinal GABA in form-deprived eyes, and with the developmental responses of the eye to these drugs. While not yet revealing clear mechanisms of operation, the nature of the GABA drug effects on the growth of both goggled and non-goggled (open) eyes, and the enrichment of GABA_{A0r} receptors in the retina, suggest that GABA pharmacology adds a useful dimension in studying retinal mechanisms that modulate eye growth and geometric form. Moreover, because the enlarged eyes receiving muscimol became myopic, while those receiving drugs with differing specificity remained emmetropic, GABA_{A} drugs appear to be useful for dissecting retinal emmetropization mechanisms.

Nevertheless, the present invention is not so limited, and is intended to include methods and compositions for controlling postnatal ocular growth and the development of ocular errors in the maturing eye of a subject, comprising altering the refraction and/or growth of the maturing eye of a subject by administering to the eye a therapeutically effective amount of at least one GABA drug or compound, including agonists or antagonists (alone or in combination with other compounds), as well as any other drug or composition, regardless of classification, that acts to alter the refractive development and/or growth of the eye. Because retinal GABA concentrations are altered in myopia, and retinal GABA influences the refractive development and growth of the eye, the present invention also alternatively conceives of another direction to alter the refractive development and growth of the eye by modulating retinal GABA levels in the maturing eye of a subject by administering to the eye to a therapeutically effective amount of at least one GABA drug or compound, including agonists or antagonists (alone or in combination with other compounds), as well as any other drug or composition, regardless of classification, that acts to correct a disorder of retinal GABA.

The disclosures of each patent, patent application and publication cited or described in this document are hereby incorporated herein by reference, in their entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

1. A method of controlling postnatal growth of an eye of a maturing animal or human, which method comprises administering GABA, or at least one agonist or antagonist thereof.

2. (canceled)

3. A method of claim 1, further comprising changing GABA in the eye or the concentration thereof.

4. The method of claim 1, wherein the postnatal growth of the eye further comprises abnormal growth.

5. The method of claim 1, wherein the postnatal growth of the eye leads to abnormal refraction.

6. The method of claim 1, comprising administering to the eye a therapeutically effective amount of a GABA drug or composition.

7. The method of claim 6, wherein administering the GABA drug or composition affects GABA receptors of types GABA_{A} , GABA_{B} or GABA_{A0r} in the eye.

8. The method of claim 6, wherein the administered drug or composition comprises at least one agonist of at least one type of GABA receptor.

9. The method of claim 6, wherein the administered drug or composition comprises at least one antagonist of at least one type of GABA receptor.

10. The method of claim 1, further comprising inhibiting or reversing myopia or its onset, or the progression of myopia in the eye of a postnatal animal or human.

11. The method of claim 10, further comprising inhibiting or reducing growth in terms of axial length or vitreous chamber depth of the eye, or in terms of equatorial expansion of the eye, or a combination thereof, thereby preventing, inhibiting or reducing myopic refraction or the onset or progression of myopia.

12. (canceled)

13. The method of claim 1, further comprising inhibiting or reversing hyperopia or its onset, or reducing the progression of hyperopia in the eye of a postnatal animal.

14. The method of claim 13, further comprising stimulating or enhancing growth in terms of axial length or vitreous chamber depth of the eye, or in terms of equatorial expansion of the eye, or a combination thereof, thereby preventing, inhibiting or reducing hyperopic refraction or reducing the progression of hyperopia.

15. (canceled)

16. The method of claim 1, further comprising inhibiting or reversing amblyopia or its onset, or reducing the progression of amblyopia in the eye of a postnatal animal or human.

17. The method of claim 1, further comprising administering to the maturing eye a therapeutically effective amount of GABA_{A} receptor agonist in a carrier or diluent buffered to a pH suitable for ocular administration.

18. The method of claim 17, wherein the GABA_{A} receptor agonist is muscimol or TACA.

19. The method of claim 1, further comprising administering to the maturing eye a therapeutically effective amount of GABA_{A} receptor antagonist in a carrier or diluent buffered to a pH suitable for ocular administration.

20. The method of claim 19, wherein the GABA_{A} receptor antagonist is SR95531 or bicuculline.

21. The method of claim 1, further comprising administering to the maturing eye a therapeutically effective amount of GABA_{A0r} receptor agonist in a carrier or diluent buffered to a pH suitable for ocular administration.

22. The method of claim 21, wherein the GABA_{A0r} receptor agonist is CACA.

23. The method of claim 1, further comprising administering to the maturing eye a therapeutically effective amount of GABA_{A0r} receptor antagonist in a carrier or diluent buffered to a pH suitable for ocular administration.

24. The method of claim 23, wherein the GABA_{A0r} receptor antagonist is TPMPA.

25. The method of claim 1, further comprising administering to the maturing eye a therapeutically effective amount of GABA_{B} receptor agonist in a carrier or diluent buffered to a pH suitable for ocular administration.

26. The method of claim 26, wherein the GABA_{B} receptor agonist is baclofen.

27. The method of claim 1, further comprising administering to the maturing eye a therapeutically effective amount of GABA_{B} receptor antagonist in a carrier or diluent buffered to a pH suitable for ocular administration.

28. The method of claim 27, wherein the GABA_{B} receptor antagonist is CGP46381, SCH50911 or 2OH-saclofen.

29-36. (canceled)

37. The method of claim 1, wherein the administering step is in vivo, thereby causing treatment of myopic hyperopic or amblyopic in the eye or eyes of the maturing animal or human.

38. The method of claim 1, wherein the administering step is in vivo, thereby further causing prevention of myopia, hyperopia or amblyopia in the eye or eyes of the maturing animal or human.

39. A method of controlling the refractive development of an eye of a maturing animal or human, which method comprises administering GABA, or at least one agonist or antagonist thereof.

40. The method of claim 39, further comprising changing GABA in the eye or the concentration thereof.

41. The method of claim 39, wherein the postnatal growth of the eye further comprises abnormal growth.

42. The method of claim 39, wherein the postnatal growth of the eye leads to abnormal refraction.

43. The method of claim 39, comprising administering to the eye a therapeutically effective amount of a GABA drug or composition.

44. The method of claim 43, wherein administering the GABA drug or composition affects GABA receptors of types GABA_{A} , GABA_{B} or GABA_{A0r} in the eye.

45. The method of claim 43, wherein the administered drug or composition comprises at least one agonist of at least one type of GABA receptor.

46. The method of claim 43, wherein the administered drug or composition comprises at least one antagonist of at least one type of GABA receptor.

47. The method of claim 39, further comprising inhibiting or reversing myopia or its onset, or the progression of myopia in the eye of a postnatal animal or human.

48. The method of claim 47, further comprising inhibiting or reducing growth in terms of axial length or vitreous chamber depth of the eye, or in terms of equatorial expansion of the eye, or a combination thereof, thereby preventing, inhibiting or reducing myopic refraction or the onset or progression of myopia.

49. The method of any one of claim 39, further comprising inhibiting or reversing hyperopia or its onset, or reducing the progression of hyperopia in the eye of a postnatal animal.

50. The method of claim 49, further comprising stimulating or enhancing growth in terms of axial length or vitreous chamber depth of the eye, or in terms of equatorial expansion of the eye, or a combination thereof, thereby preventing, inhibiting or reducing hyperopic refraction or reducing the progression of hyperopia.

51. The method of claim 39, further comprising inhibiting or reversing amblyopia or its onset, or reducing the progression of amblyopia in the eye of a postnatal animal or human.

52. The method of any one of claim 39, further comprising administering to the maturing eye a therapeutically effective amount of GABA_{A} receptor agonist in a carrier or diluent buffered to a pH suitable for ocular administration.

53. The method of claim 52, wherein the GABA_{A} receptor agonist is muscimol or TACA.

54. The method of claim 39, further comprising administering to the maturing eye a therapeutically effective amount of GABA_{A} receptor antagonist in a carrier or diluent buffered to a pH suitable for ocular administration.

55. The method of claim 54, wherein the GABA_{A} receptor antagonist is SR95531 or bicuculline.

56. The method of claim 39, further comprising administering to the maturing eye a therapeutically effective amount of GABA_{A0r} or receptor agonist in a carrier or diluent buffered to a pH suitable for ocular administration.

57. The method of claim 56, wherein the GABA_{A0r} receptor agonist is CACA.

58. The method of claim 39, further comprising administering to the maturing eye a therapeutically effective amount of GABA_{A0r} receptor antagonist in a carrier or diluent buffered to a pH suitable for ocular administration.

59. The method of claim 58, wherein the GABA_{A0r} receptor antagonist is TPMPA.

60. The method of claim 39, further comprising administering to the maturing eye a therapeutically effective amount of GABA_{B} receptor agonist in a carrier or diluent buffered to a pH suitable for ocular administration.

61. The method of claim 60, wherein the GABA_{B} receptor agonist is baclofen.

62. The method of claim 39, further comprising administering to the maturing eye a therapeutically effective amount of GABA_{B} receptor antagonist in a carrier or diluent buffered to a pH suitable for ocular administration.

63. The method of claim 62, wherein the GABA_{B} receptor antagonist is CGP46381, SCH50911 or 2OH-saclofen.

64. The method of claim 39, wherein the administering step is in vivo, thereby causing treatment of myopic hyperopic or amblyopic in the eye or eyes of the maturing animal or human.

65. The method of claim 39, wherein the administering step is in vivo, thereby further causing prevention of myopia, hyperopia or amblyopia in the eye or eyes of the maturing animal or human.

66. A method of controlling the postnatal growth of the eye of a maturing animal or human, comprising administering to the eye of the animal or human an effective amount of a neurochemical, or the agonist or the antagonist thereof, thereby modulating the presence of GABA, or its agonist or antagonist.

67. A method of detecting the effect of one or more GABA drugs or compounds affecting the ocular growth of a maturing eye of a postnatal animal comprising:
administering to a first animal eye a therapeutically effective amount of a retinal GABA receptor agonist or antagonist in a carrier or diluent buffered to a pH suitable for ocular administration;
detecting change in growth in the axial or equatorial direction, or both, in the first eye;
administering to a second animal eye a control agent, comprising the carrier or diluent used with the retinal GABA receptor agonist or antagonist in the first eye;
detecting effects of the control agent on the second eye; and
comparing the change in growth in the first eye with the effects of the control agent on the second eye;
wherein the first eye is open or covered, as by suturing closed or goggling, and
wherein the second eye is the same (open or covered) as the first eye.

68. The method of claim 67, wherein the first animal eye and the second animal eye are in the same animal.

69. The method of claim 67, wherein the first animal eye and the second animal eye are in different animals.

70. A composition of matter useful for controlling postnatal growth of an eye of a maturing animal or human, wherein GABA is changed by said composition during postnatal maturation of the eye.
